(19) United States
(12) Patent Application Publication
(10) Pub. No.: US 2026/0345314 A1
(43) Pub. Date: Nov. 5, 2026

(54) BONE SCREW AND METHOD OF REPAIRING SOFT TISSUE

(71) Applicant: AEVUMED, INC., Malvern, PA (US)

(72) Inventors: Robert P. Douglass, Bryn Mawr, PA (US); Miles O. Curtis, Bryn Mawr, PA (US); Saif Khalil, Malvern, PA (US); lan McGorrey, Malvern, PA (US); Scott Steinmann, Malvern, PA (US); William Geissler, Malvern, PA (US)

(21) Appl. No.: 19/454,625

(22) Filed: Jan. 21, 2026

Related U.S. Application Data
(60) Provisional application No. 63/748,290, filed on Jan. 22, 2025.

(51) Int. Cl. A61F 2/08; A61B 17/86

(52) U.S. Cl. CPC A61F 2/0811; A61B 17/8635; A61B 17/8645; A61F 2002/0852; A61F 2002/0888

(57) ABSTRACT

A bone screw includes an exterior wall having a proximal end and a distal end, and a central channel extending along a central axis of the bone screw and having a proximal opening. A first set of protrusions are disposed on the exterior wall, the first set of protrusions tapering towards the central axis in a distal direction. A second set of protrusions are disposed on the exterior wall, the set plurality of protrusions tapering towards the central axis in a distal direction. A forked distal end has a first leg, a second leg and a fork opening therebetween. A concave exterior wall portion extends at least partially along the central axis. A traverse opening extends from the concave exterior wall portion through to the opposing exterior wall, traversing the central channel. A method for treating soft tissue is also described.

### CROSS-REFERENCE TO RELATED APPLICATIONS

This application claims priority to U.S. provisional application No. 63/748,290 filed on Jan. 22, 2025 incorporated herein by reference in its entirety.

### BACKGROUND OF THE INVENTION

Bone screws such as tendon screws, also known as interference screws, are used in orthopedic surgeries for soft tissue repair, such as securing tendons or ligaments to bone. These screws are typically made from biocompatible materials such as titanium or biodegradable polymers. The design often includes threads or barbed protrusions to provide a strong grip and ensure stable fixation. Tendon screws are most commonly used in reconstructive procedures, such as rotator cuff repairs, anterior cruciate ligament (ACL) repairs, or posterior cruciate ligament (PCL) repairs, where a tendon or graft needs to be anchored within a drilled bone tunnel. By holding the graft securely in place, these screws facilitate proper contact and integration of the tendon or graft with the surrounding bone, ultimately restoring joint stability and function and providing an optimal tendon and bone interface for healing. Their use allows for a more precise and durable reconstruction, supporting patients in regaining mobility and reducing the risk of reinjury.

For example, in a rotator cuff repair procedure where a tendon screw is used to repair a tendon, the surgery aims to reattach a torn tendon in the shoulder to its original attachment site on the humerus (upper arm bone) using a secure fixation technique. The surgeon begins by making a small incision or using arthroscopic tools to access the shoulder joint. The torn edges of the tendon are carefully prepared, which may include trimming any frayed tissue to create a clean attachment surface and whip stitching the end of the tendon with a suture to grasp it. The bone at the tendon's original attachment site may be debrided to expose healthy, bleeding bone, which promotes healing.

A bone tunnel or pilot hole is drilled at the repair site. The tendon is positioned over the bone, and a tendon screw (often an interference screw) is inserted into the drilled hole alongside the tendon. This screw compresses the tendon against the bone to provide a firm fixation. Once the tendon is securely anchored, the surgeon tests the repair for strength and range of motion before closing the incision or arthroscopic portals. Post-surgery, the patient undergoes a period of immobilization followed by physical therapy to promote healing and restore shoulder function.

There are however disadvantages to conventional tendon screw devices and methods of treatment. Tendon screws have propensity to tear tendon when screw and tendon are inserted into bore hole. Conventional tendon screw designs provide little ability for surgeons to adequately secure the tendon to bone, nor do they provide flexibility for setting preferred suture tension.

Accordingly, there is a need in the art for a tendon anchor and method of insertion for providing a secure attachment of the tendon within the bone hole, minimizing the chances of the inserted screw tearing the tendon, providing an ability to apply preferred suture tensions, and overall providing an optimal tendon to bone interface for healing. Embodiments of the tendon screw and method of repairing a tendon described herein fit this need.

### SUMMARY OF THE INVENTION

In one embodiment, a bone screw includes an exterior wall having a proximal end and a distal end; a central channel extending along a central axis of the bone screw and having a proximal opening; a first set of protrusions disposed on the exterior wall, the first set of protrusions tapering towards the central axis in a distal direction; a second set of protrusions disposed on the exterior wall, the second set of protrusions tapering towards the central axis in a distal direction; a forked distal end having a first leg, a second leg and a fork opening therebetween; a concave exterior wall portion extending at least partially along the central axis; and a traverse opening extending from the concave exterior wall portion through to the opposing exterior wall, traversing the central channel. In one embodiment, the first and second set of protrusions are configured to form an interference fit with a bone hole. In one embodiment, the second set of tapered protrusions extend further away from the exterior wall than the first set of protrusions. In one embodiment, at least one of the first and second set of protrusions has a top surface substantially perpendicular to the central axis. In one embodiment, the second set of protrusions are disposed radially around the exterior wall at a common distance from a distal tip of the bone screw. In one embodiment, the second set of protrusions are separated by concave gaps. In one embodiment, the second set of protrusions comprises at least a first and second protrusion having a gap disposed above one end of the traverse opening. In one embodiment, at least one of the second set of protrusions is disposed on the concave exterior wall portion. In one embodiment, a first end of the traverse opening is disposed on the concave exterior wall. In one embodiment, a second end of the traverse opening opposes the first end of the traverse opening and is disposed at least partially on at least one of the first set of protrusions. In one embodiment, the bone screw includes a first inserter channel having a first proximal opening on a proximal surface of the bone screw, and a second inserter channel having a second proximal opening on the proximal surface of the bone screw. In one embodiment, a bone screw system includes the bone and a set screw configured to advance distally within the central channel. In one embodiment, the central channel has a first threaded surface, the set screw has a second threaded surface configured to interface with the first threaded surface to move the set screw along the central axis within the central channel. In one embodiment, a method for repairing soft tissue includes providing the bone screw; attaching a suture to the soft tissue; passing the suture through the fork opening and the traverse opening; and advancing the set screw distally to apply a retaining force on the suture. In one embodiment, the bone screw system includes an outer insertion member having a forked distal end and an insertion member lumen; and an inner insertion member having a distal tip configured to interface with a proximal structure of the set screw; where the inner insertion member is configured to advance through and beyond a distal opening of the insertion member lumen and freely rotate within the insertion member lumen.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is perspective view of a bone anchor with the set screw inserted according to one embodiment, FIG. 1B is an alternate perspective view of the bone anchor of FIG. 1A with the set screw inserted, FIG. 1C is an alternate perspective view of the bone anchor of FIG. 1A with the set screw removed and including a partial alternate top view, and FIG. 1D is a perspective view of a set screw shown in isolation according to one embodiment.

FIGS. 2A through 2D show perspective views of a tendon repair procedure according to one embodiment.

FIGS. 3A through 3G show various perspective views of an outer and inner insertion member, including the inner insertion member inserted into the outer insertion member (FIG. 3A), a magnified distal tip view of the inner insertion member inserted into the outer insertion member (FIG. 3B), a magnified distal tip view of the inner insertion member inserted into the outer insertion member while engaged with a bone anchor and set screw (FIG. 3C), the outer insertion member in isolation (FIG. 3D), a magnified distal view of the outer insertion member in isolation (FIG. 3E), the inner insertion member in isolation (FIG. 3F), and a magnified distal view of the inner insertion member in isolation (FIG. 3G).

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in bone anchors and methods of treating soft tissue. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a bone screw and method of treating soft tissue.

Embodiments of the bone screw described herein provides several advantages over conventional bone screws. Advantages include less whipstitching required, no tendon wrapping required via the push-in design, less likely to cause tendon tearing, adjustable tendon tensioning pre-insertion, small drill hole sizes can be implemented, and significantly improved bone and tendon contact.

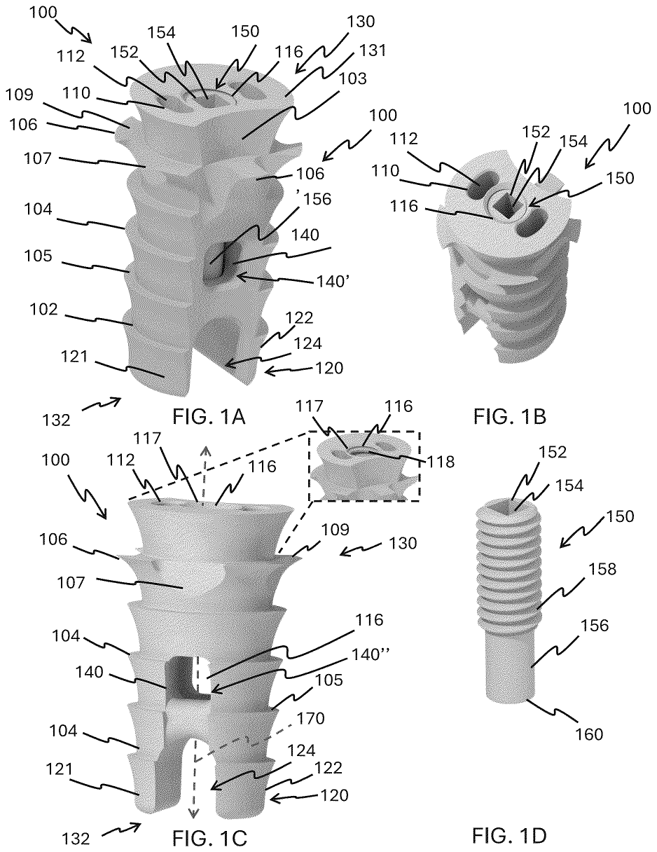

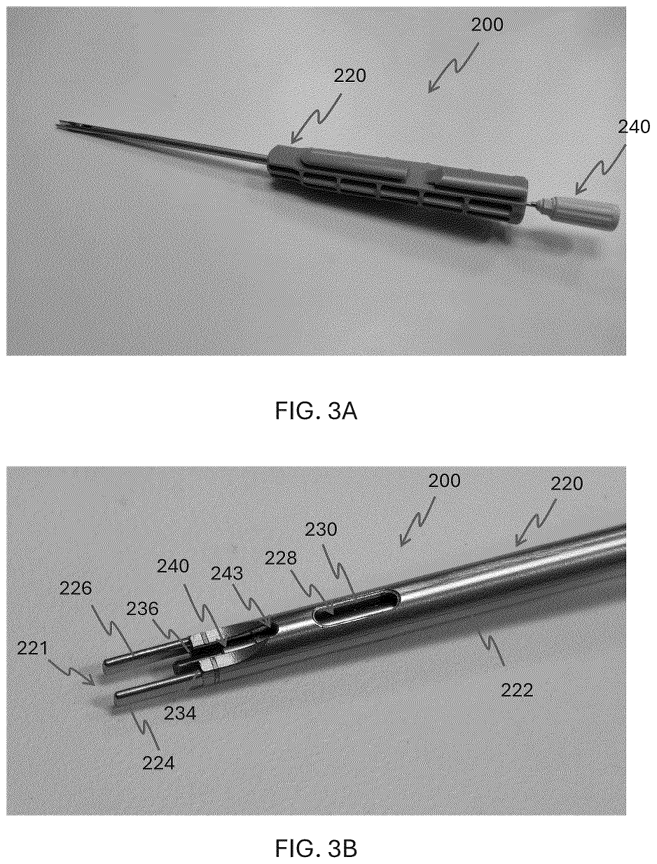

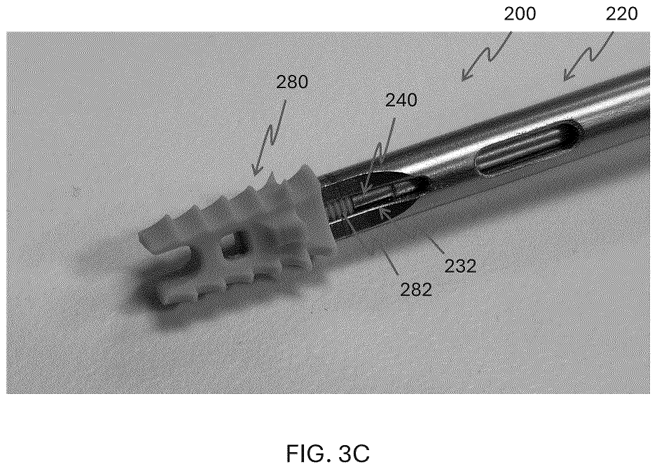

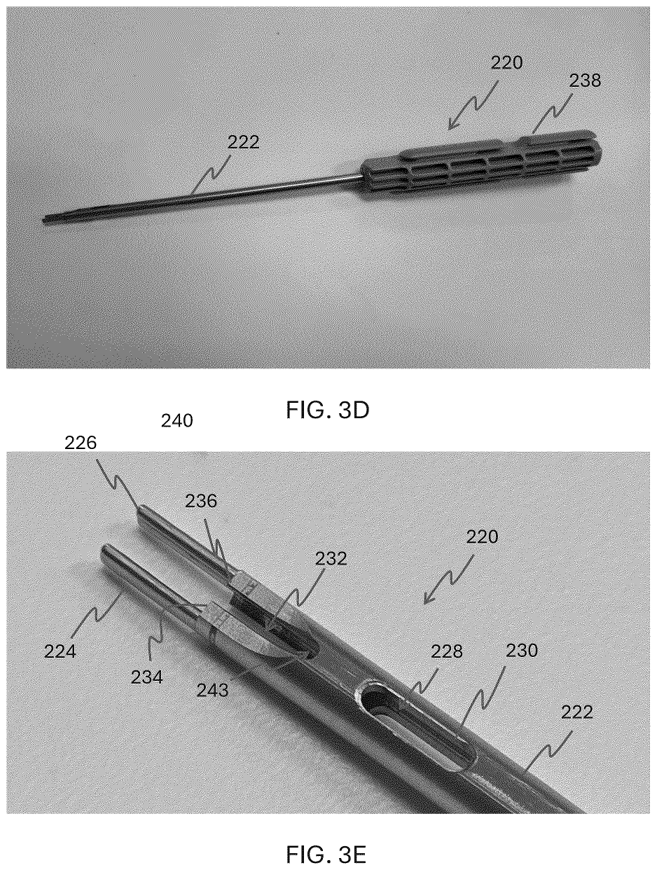

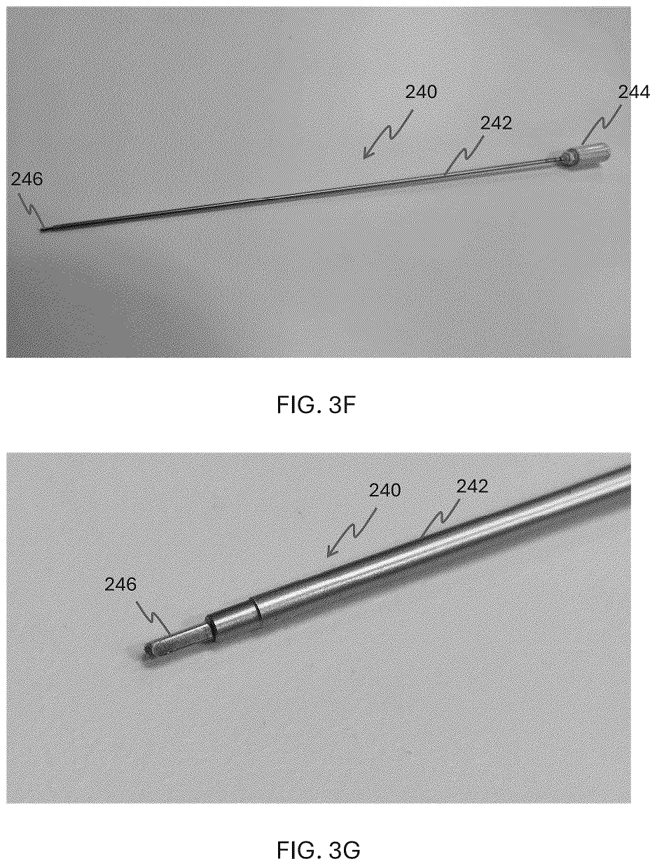

With reference now to FIG. 1A-1D, a bone screw 100 is shown according to one embodiment. The bone screw 100 has an exterior wall 102 extending between a proximal end 130 and a distal end 132 of the bone screw 100. A central channel 116 extends along a central axis 170 of the bone screw 100. The central channel 116 has a proximal opening 117 and extends distally down to a central channel bottom surface 119 (shown in the cutaway view of FIG. 3B). A first set of protrusions 104 are disposed on the exterior wall 102, tapering towards the central axis 170 in a distal direction. A second set of protrusions 106 are disposed on the exterior wall 102, tapering towards the central axis 170 in a distal direction. The first and second sets of protrusions 104, 106 are configured to form an interference fit with a bone hole. A forked distal end 120 having a first leg 121, a second leg 122 and a fork opening 124 between the first and second legs 121, 122. The fork opening 124 can be for example an inverted U shape with smooth sidewalls.

A concave exterior wall portion 103 extends at least partially along the bone screw 100. A traverse opening 116 extends from one opening 140′ on the concave exterior wall portion 103 through to a second opposing opening 140″, traversing the central channel 116. The second set of tapered protrusions 106 extend further away from the exterior wall 102 than the first set of protrusions 104. The first and second sets of protrusions 104, 106 have a top surface substantially perpendicular to the central axis 170. The second set of protrusions 106 are disposed radially around the exterior wall 102 at a common distance from a distal tip of the bone screw 100. The second set of protrusions 106 are separated by concave gaps. The second set of protrusions 106 includes at least a first and second protrusion having a gap 107 disposed above one end of the traverse opening on a side of the bone anchor 100 opposing the concave exterior wall portion 103. At least one of the second set of protrusions 106 is disposed on the concave exterior wall portion 103. The first end 140′ of the traverse opening 140 is disposed on the concave exterior wall 103, and the second end 140″ of the traverse opening 140 opposes the first end 140′ of the traverse opening 140 and is disposed at least partially on at least one of the first set of protrusions 104.

The bone screw includes a first inserter channel having a first proximal opening on a proximal surface of the bone screw, and a second inserter channel having a second proximal opening on the proximal surface of the bone screw. The bone screw system further includes a set screw 150 configured to advance distally within the central channel 116. The set screw 150 includes a central channel 154 having a proximal set screw opening 152. The proximal set screw opening 152 may include a geometry such as a square or another shape to mate with the head of an insertion tool configured to rotate or advance the set screw 150 within the bone anchor's central channel 116. An inserter tool has an outer component with a forked distal end that interfaces with the inserter channels 112, and an inner component disposed within a lumen of the outer component, freely rotatable and with a distal end that interfaces with the proximal set screw opening 152 to turn the set screw 150 within the central channel 116. The central channel 116 has a first threaded surface 118, and the set screw 150 has a second threaded surface 158 configured to interface with the first threaded surface 118 to move the set screw 150 along the central axis 170 within the central channel 116.

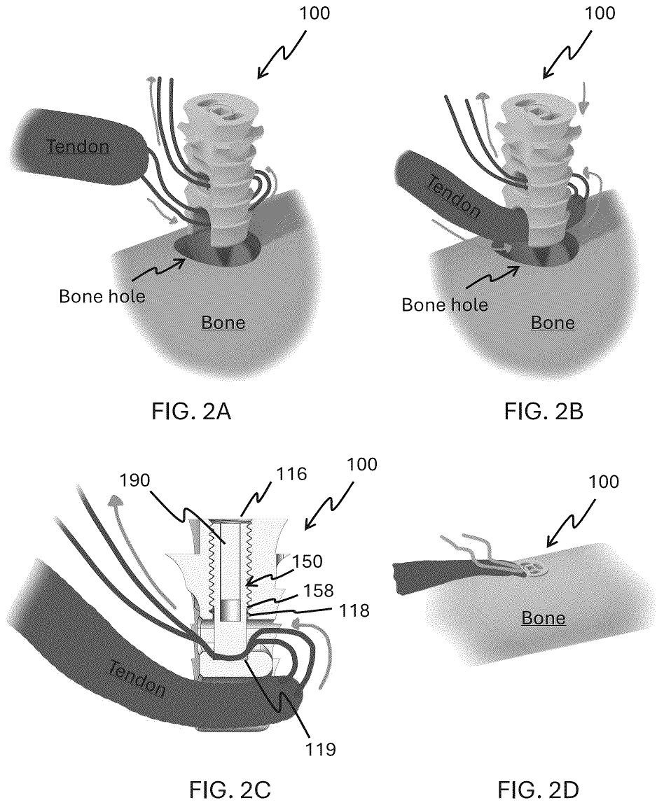

A method for repairing soft tissue is now described according to one embodiment with reference to FIGS. 2A-2D. In one embodiment, the method includes attaching a suture to the soft tissue, passing the suture through the fork opening and the traverse opening (FIGS. 2A and 2B), and advancing the set screw distally to apply a retaining force on the suture (FIG. 2C). The bone screw can then be advanced fully down into the bone hole.

With reference now to FIGS. 3A-3G, in one embodiment, the bone screw system includes an insertion device 200 including an outer insertion member 220 having a pair of forks 224, 226 at a forked distal end 221 and an insertion member lumen 228. The insertion member lumen 228 can connect to one or more windows 230 in a sidewall 222 of the lumen 228. A distal end of the outer insertion member 220 may have a slot 232 at a distal opening 243 of the insertion member lumen 228. The pair of forks 224, 226 at the forked distal end 221 may terminate proximally at a corresponding pair of shoulders 234, 236 that interface with a proximal face of the bone screw 280. A handle 238 at a proximal end of the outer insertion member 220 includes a larger structure and recessed portions for manual grip. An inner insertion member 240 has a distal tip 246 configured to interface with a proximal structure of the set screw 282 and a proximal handle 244. The inner insertion member 240 can advance through and freely rotate within the lumen 228 of the outer insertion member 220. The distal tip 246 of the inner insertion member 240 can engage the proximal opening of the set screw 282 to advance or retract the set screw 282 with rotational motion while the forked distal end 221 of the outer insertion member 220 keeps the bone screw 280 in place and prevents it from turning or moving. The inner insertion member 240 is configured to advance through a distal opening 243 of the insertion member lumen 228 and freely rotate within the insertion member lumen 230, and movement of the inner insertion member 240 can be viewed through the window 230 and within the slot 232.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A bone screw comprising:
an exterior wall having a proximal end and a distal end;
a central channel extending along a central axis of the bone screw and having a proximal opening;
a first plurality of protrusions disposed on the exterior wall, the first plurality of protrusions tapering towards the central axis in a distal direction;
a second plurality of protrusions disposed on the exterior wall, the second plurality of protrusions tapering towards the central axis in a distal direction;
a forked distal end having a first leg, a second leg and a fork opening therebetween;
a concave exterior wall portion extending at least partially along the central axis; and
a traverse opening extending from the concave exterior wall portion through to the opposing exterior wall, traversing the central channel.

2. The bone screw of claim 1, wherein the first and second plurality of protrusions are configured to form an interference fit with a bone hole.

3. The bone screw of claim 1, wherein the second plurality of tapered protrusions extend further away from the exterior wall than the first plurality of protrusions.

4. The bone screw of claim 1, wherein at least one of the first and second plurality of protrusions has a top surface substantially perpendicular to the central axis.

5. The bone screw of claim 1, wherein the second plurality of protrusions are disposed radially around the exterior wall at a common distance from a distal tip of the bone screw.

6. The bone screw of claim 1, wherein the second plurality of protrusions are separated by concave gaps.

7. The bone screw of claim 1, wherein the second plurality of protrusions comprises at least a first and second protrusion having a gap disposed above one end of the traverse opening.

8. The bone screw of claim 1, wherein at least one of the second plurality of protrusions is disposed on the concave exterior wall portion.

9. The bone screw of claim 1, wherein a first end of the traverse opening is disposed on the concave exterior wall.

10. The bone screw of claim 9, wherein a second end of the traverse opening opposes the first end of the traverse opening and is disposed at least partially on at least one of the first plurality of protrusions.

11. The bone screw of claim 1 further comprising:
a first inserter channel having a first proximal opening on a proximal surface of the bone screw, and a second inserter channel having a second proximal opening on the proximal surface of the bone screw.

12. A bone screw system comprising:
the bone screw of claim 1; and
a set screw configured to advance distally within the central channel.

13. The bone screw system of claim 12, wherein the central channel has a first threaded surface, the set screw has a second threaded surface configured to interface with the first threaded surface to move the set screw along the central axis within the central channel.

14. A method for repairing soft tissue comprising:
providing the bone screw system of claim 13;
attaching a suture to the soft tissue;
passing the suture through the fork opening and the traverse opening; and
advancing the set screw distally to apply a retaining force on the suture.

15. The bone screw system of claim 12 further comprising:
an outer insertion member having a forked distal end and an insertion member lumen; and
an inner insertion member having a distal tip configured to interface with a proximal structure of the set screw;
wherein the inner insertion member is configured to advance through and beyond a distal opening of the insertion member lumen and freely rotate within the insertion member lumen.
